(19) United States
(12) Patent Application Publication
(10) Pub. No.: US 2003/0228589 A1
(43) Pub. Date: Dec. 11, 2003

(54) Polymerase compositions

(76) Inventors: Diane R. Bond-Nutter, Oakland, CA (US); Cheryl R. Heiner, La Honda, CA (US); James E. Rozzelle, San Francisco, CA (US)

Correspondence Address: Finnegan, Henderson, Farabow,, Garrett & Dunner, L.L.P., 1300 I Street, N.W., Washington, DC, 20005-3315, US

(21) Appl. No.: 10/365,032

(22) Filed: Feb. 11, 2003

Related U.S. Application Data
(60) Provisional application No. 60/357,087, filed on Feb. 12, 2002.

(51) Int. Cl. C12Q 1/68; C12P 19/34; C12N 1/21

(52) U.S. Cl. 435/6; 435/91.1; 435/252.3

(57) ABSTRACT

Compositions comprising at least two polymerases are provided. Methods for producing primer extension products using at least two polymerases are also provided. Kits for producing primer extension products comprising at least two polymerases are also provided.

This application claims priority to U.S. Provisional Application Serial No. 60/357,087, filed Feb. 12, 2002, which is incorporated herein by reference for any purpose.

### FIELD OF THE INVENTION

The present invention generally relates to compositions comprising different polymerases and methods that employ such compositions.

### BACKGROUND OF THE INVENTION

DNA polymerases are enzymes that synthesize DNA molecules from deoxynucleotide triphosphates (dNTPs) using a template DNA strand and a complementary oligonucleotide primer annealed to a portion of the template DNA strand. A detailed description of certain DNA polymerases and their characterization can be found, e.g., in Kornberg, DNA Replication Second Edition, W. H. Freeman (1989).

DNA polymerases have a variety of uses in molecular biology techniques. Such techniques include primer extension reactions, DNA sequencing, and nucleic acid amplification techniques such as the polymerase chain reaction (PCR).

### SUMMARY OF THE INVENTION

In certain embodiments, a composition comprising at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G) is provided.

In certain embodiments, a composition comprising Thermus thermophilus GK24 (G46D; F669Y) and Thermus oshimai (G43D; F665Y) is provided.

In certain embodiments, the composition further comprises at least one primer. In certain embodiments, the composition further comprises at least one extendable nucleotide. In certain embodiments, the composition further comprises at least one terminator.

In certain embodiments, a method of generating at least one primer extension product is provided. In certain embodiments, methods comprise forming a reaction composition comprising at least one target nucleic acid template, at least one primer, at least one extendable nucleotide, and at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G); and incubating the composition under appropriate conditions to generate at least one primer extension product.

In certain embodiments, a method of generating at least one primer extension product comprises forming a reaction composition comprising at least one target nucleic acid template, at least one primer, at least one extendable nucleotide, and Thermus thermophilus GK24 (G46D; F669Y) and Thermus oshimai (G43D; F665Y); and incubating the composition under appropriate conditions to generate at least one primer extension product.

In certain embodiments, the method further comprises separating the at least one primer extension product. In certain embodiments, the method further comprises detecting the at least one primer extension product.

In certain embodiments, methods of sequencing a target nucleic acid template are provided. In certain embodiments, a method of sequencing a target nucleic acid template comprises forming a reaction composition comprising at least one target nucleic acid template, at least one primer, at least one extendable nucleotide, at least one terminator, and at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G); incubating the composition under appropriate conditions to generate at least one primer extension product; separating the at least one primer extension product; detecting the at least one primer extension product; and determining the sequence of the target nucleic acid template.

In certain embodiments, a kit is provided. In certain embodiments, the kit comprises at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G).

In certain embodiments, the kit comprises Thermus thermophilus GK24 (G46D; F669Y) and Thermus oshimai (G43D; F665Y).

In certain embodiments, the kit further comprises at least one terminator. In certain embodiments, the kit further comprises at least one extendable nucleotide. In certain embodiments, the kit further comprises at least one primer.

### DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Definitions

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2 -dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures:

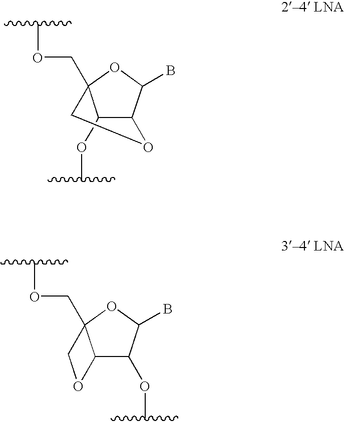

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

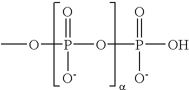

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

An “extendable nucleotide” is a nucleotide which is: (i) capable of being enzymatically or synthetically incorporated onto the terminus of a polynucleotide chain, and (ii) capable of supporting further enzymatic or synthetic extension. Extendable nucleotides include nucleotides that have already been enzymatically or synthetically incorporated into a polynucleotide chain, and have either supported further enzymatic or synthetic extension, or are capable of supporting further enzymatic or synthetic extension. Extendable nucleotides include, but are not limited to, nucleotide 5′-triphosphates, e.g., dNTP and NTP, phosphoramidites suitable for chemical synthesis of polynucleotides, and nucleotide units in a polynucleotide chain that have already been incorporated enzymatically or chemically.

The term “nucleotide terminator” or “terminator”, as used herein, refers to an enzymatically-incorporable nucleotide, which does not support incorporation of subsequent nucleotides in a primer extension reaction. A terminator is therefore not an extendable nucleotide. In certain embodiments, terminators are those in which the nucleotide is a purine, a 7-deaza-purine, a pyrimidine, or a nucleotide analog, and the sugar moiety is a pentose which includes a 3′-substituent that blocks further synthesis, such as a dideoxynucleotide triphosphate (ddNTP). In certain embodiments, substituents that block further synthesis include, but are not limited to, amino, deoxy, halogen, alkoxy and aryloxy groups. Exemplary terminators include, but are not limited to, those in which the sugar-phosphate ester moiety is 3′-(C1-C6)alkylribose-5′-triphosphate, 2′-deoxy-3′-(C1-C6)alkylribose-5′-triphosphate, 2′-deoxy-3′-(C1-C6)alkoxyribose-5-triphosphate, 2′-deoxy-3′-(C5-C14)aryloxyribose-5′-triphosphate, 2′-deoxy-3′-haloribose-5′-triphosphate, 2′-deoxy-3′-aminoribose-5′-triphosphate, 2′,3′-dideoxyribose-5′-triphosphate or 2′,3′-didehydroribose-5′-triphosphate. Terminators include, but are not limited to, T terminators, including ddTTP and dUTP, which incorporate opposite an adenine, or adenine analog, in a template; A terminators, including ddATP, which incorporate opposite a thymine, uracil, or an analog of thymine or uracil, in the template; C terminators, including ddCTP, which incorporate opposite a guanine, or guanine analog, in the template; and G terminators, including ddGTP and ddITP, which incorporate opposite a cytosine, or cytosine analog, in the template.

The term “label” refers to any moiety which can be attached to a molecule and: (i) provides a detectable signal; (ii) interacts with a second label to modify the detectable signal provided by the second label, e.g. FRET (Fluorescent Resonance Energy Tansfer); (iii) stabilizes hybridization, e.g., duplex formation; or (iv) provides a member of a binding complex or affinity set, e.g., affinity, antibody/antigen, ionic complexation, hapten/ligand, e.g. biotin/avidin. Labeling can be accomplished using any one of a large number of known techniques employing known labels, linkages, linking groups, reagents, reaction conditions, and analysis and purification methods. Labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful for labelling biomolecules include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes (ETFDs), comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent label capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. Labels also include, but are not limited to, semiconductor nanocrystals, or quantum dots (see, e.g., U.S. Pat. Nos. 5,990,479 and 6,207,392 B1; Han et al. Nature Biotech. 19: 631-635).

A class of labels are hybridization-stabilizing moieties which serve to enhance, stabilize, or influence hybridization of duplexes, e.g. intercalators, minor-groove binders, and cross-linking functional groups (see, e.g., Blackburn, G. and Gait, M. Eds. “DNA and RNA structure” in Nucleic Acids in Chemistry and Biology, 2^{nd} Edition, (1996) Oxford University Press, pp.15-81). Yet another class of labels effect the separation or immobilization of a molecule by specific or non-specific capture, for example biotin, digoxigenin, and other haptens (see, e.g., Andrus, A. “Chemical methods for 5′ non-isotopic labeling of PCR probes and primers” (1995) in PCR 2: A Practical Approach, Oxford University Press, Oxford, pp.39-54). Non-radioactive labelling methods, techniques, and reagents are reviewed in: Non-Radioactive Labelling, A Practical Introduction, Garman, A. J. (1997) Academic Press, San Diego.

Labels may be “detectably different”, which means that they are distinguishable from one another by at least one detection method. Detectably different labels include, but are not limited to, labels that emit light of different wavelengths, labels that absorb light of different wavelengths, labels that have different fluorescent decay lifetimes, labels that have different spectral signatures, labels that have different radioactive decay properties, labels of different charge, and labels of different size.

The term “labeled terminator”, as used herein, refers to a terminator that is physically joined to a label. The linkage to the label is at a site or sites on the terminator that do not prevent the incorporation of the terminator by a polymerase into a polynucleotide.

As used herein, the term “target nucleic acid template” refers to a nucleic acid sequence that serves as a template for a primer extension reaction. Target nucleic acid templates include, but are not limited to, genomic DNA, including mitochondrial DNA and nucleolar DNA, cDNA, synthetic DNA, plasmid DNA, yeast artificial chromosomal DNA (YAC), bacterial artificial chromosomal DNA (BAC), and other extrachromosomal DNA, and primer extension products. Target nucleic acid templates also include, but are not limited to, RNA, synthetic RNA, mRNA, tRNA, and analogs of both RNA and DNA, such as peptide nucleic acids (PNA).

Different target nucleic acid templates may be different portions of a single contiguous nucleic acid or may be on different nucleic acids. Different portions of a single contiguous nucleic acid may overlap.

“Primer” as used herein refers to a polynucleotide or oligonucleotide that has a free 3′-OH (or functional equivalent thereof) that can be extended by at least one nucleotide in a primer extension reaction catalyzed by a polymerase. In certain embodiments, primers may be of virtually any length, provided they are sufficiently long to hybridize to a polynucleotide of interest in the environment in which primer extension is to take place. In certain embodiments, primers are at least 14 nucleotides in length. Primers may be specific for a particular sequence, or, alternatively, may be degenerate, e.g., specific for a set of sequences.

The terms “primer extension” and “primer extension reaction” are used interchangeably, and refer to a process of adding one or more nucleotides to a nucleic acid primer, or to a primer extension product, using a polymerase, a template, and one or more nucleotides.

A “primer extension product” is produced when one or more nucleotides has been added to a primer in a primer extension reaction. A primer extension product may serve as a target nucleic acid template in subsequent extension reactions. A primer extension product may include a terminator.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Polynucleotides may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

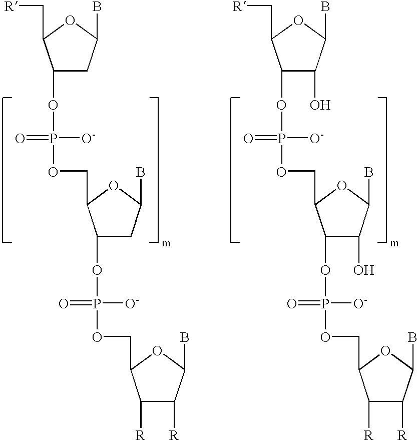

wherein each B is independently the base moiety of a nucleotide, e.g., a purine, a 7-deazapurine, a pyrimidine, or an analog thereof; each m defines the length of the respective nucleic acid and can range from zero to thousands, tens of thousands, or even more; each R is independently selected from the group comprising hydrogen, hydroxyl, halogen, —R″, —OR−, and —NR″R″, where each R″ is independently (C₁-C₆) alkyl or (C₅-C1₄) aryl, or two adjacent Rs may be taken together to form a bond such that the ribose sugar is 2′,3′-didehydroribose, and each R′ may be independently hydroxyl or

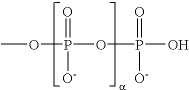

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a polynucleotide that contains at least one nucleotide analog and/or at least one phosphate ester analog and/or at least one pentose sugar analog. Also included within the definition of polynucleotide analogs are polynucleotides in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. Base-stacking and hydrophobic interactions may also contribute to duplex stability.

The term “variant” as used herein refers to any alteration of a protein, including, but not limited to, changes in amino acid sequence, substitutions of one or more amino acids, addition of one or more amino acids, deletion of one or more amino acids, and alterations to the amino acids themselves. In certain embodiments, the changes involve conservative amino acid substitutions. Conservative amino acid substitution may involve replacing one amino acid with another that has, e.g., similar hydrophobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

Alterations to the amino acids may include, but are not limited to, glycosylation, methylation, phosphorylation, biotinylation, and any covalent and noncovalent additions to a protein that do not result in a change in amino acid sequence. “Amino acid” as used herein refers to any amino acid, natural or nonnatural, that may be incorporated, either enzymatically or synthetically, into a polypeptide or protein.

“Thermostable”, as used herein, refers to a polymerase that remains active at a temperature greater than about 37° C. In certain embodiments, the nucleic acid polymerases of the invention remain active at a temperature greater than about 42° C. In certain embodiments, the nucleic acid polymerases of the invention remain active at a temperature greater than about 50° C. In certain embodiments, the nucleic acid polymerases of the invention remain active at a temperature greater than about 60° C. In certain embodiments, the nucleic acid polymerases of the invention remain active at a temperature greater than about 70° C.

As used herein, a “unit” of polymerase is defined as the amount of polymerase that will catalyze the incorporation of 10 nmoles of total nucleotide into acid-insoluble form in 30 minutes at 74° C.

As used herein, the “ratio” of one polymerase to another in a composition is determined based on the percentage of the units of the polymerase when the polymerase is used alone. Thus, as a nonlimiting example, if the ratio is 60:40, one calculates the amounts of polymerase A and polymerase B as follows. In this example, polymerase A, when used as the only polymerase in a composition, is used at a concentration of 10 units per 20 μl reaction. In this example, polymerase B, when used as the only polymerase in a composition, is used at a concentration of 20 units per 20 μl reaction. If polymerase A and polymerase B are used together in a 20 μl composition at a ratio of 60:40, then the composition contains 6 units of polymerase A (60% of 10 units) and 8 units of polymerase B (40% of 20 units).

As used herein, “mobility-dependent analysis technique” or “MDAT” means an analytical technique based on differential rates of migration among different analyte types. In certain embodiments, the primer extension products may be separated based on, e.g., mobility, molecular weight, length, sequence, and/or charge. Any method that allows two or more nucleic acid sequences in a mixture to be distinguished, e.g., based on mobility, length, molecular weight, sequence and/or charge, is within the scope of the invention. Exemplary mobility-dependent analysis techniques include, without limitation, electrophoresis, including gel or capillary electrophoresis; chromatography, including as HPLC; mass spectroscopy, including MALDI-TOF; sedimentation, including gradient centrifugation; gel filtration; field-flow fractionation; multi-stage extraction techniques; and the like. In certain embodiments, the MDAT is electrophoresis or chromatography.

Certain Exemplary Embodiments of the Invention

In certain embodiments, the present invention is directed to compositions and methods for generating at least one primer extension product. According to certain embodiments, the present invention provides methods for generating a primer extension product using at least two polymerases. In certain embodiments, the methods employ compositions comprising at least one target nucleic acid template, at least one primer, at least one extendable nucleotide, and at least two polymerases. In certain embodiments, a duplex (double stranded polynucleotide) is formed between a target nucleic acid template and a primer in the composition. In certain embodiments, the primer hybridizes to a predetermined location on the target nucleic acid template.

The composition is then incubated under appropriate reaction conditions, such that one or more extendable nucleotides are incorporated sequentially onto the 3′ end of the primer. In certain embodiments, the primer extension products generated by the primer extension reaction may then be separated based on size.

Polymerases may or may not be thermostable. In certain embodiments, polymerases have mutations that reduce discrimination against 3′-dideoxynucleotide terminators as compared with nucleotide triphosphates. In certain embodiments, a polymerase bearing one or more of these mutations may incorporate 3′-deoxynucleotide terminators with greater efficiency than does the wild type polymerase (see, e.g., U.S. Pat. No. 5,885,813 and U.S. Pat. No. 6,265,193, which are herein incorporated by reference for any purpose). In certain embodiments, mutations that reduce discrimination against 3′-dideoxynucleotide terminators are in the nucleotide-binding region of the polymerase. In certain embodiments, the nucleotide-binding region is located from about amino acid 520 to about amino acid 832 of the polymerase.

In certain embodiments, polymerases have mutations that reduce discrimination against fluorescent-labeled nucleotides. In certain embodiments, a polymerase bearing one or more of these mutations may incorporate fluorescent-labeled nucleotides with greater efficiency than does the wild type polymerase (see, e.g., U.S. Pat. No. 5,885,813 and U.S. Pat. No. 6,265,193, which are herein incorporated by reference for any purpose). In certain embodiments, mutations that reduce discrimination against fluorescent-labeled nucleotides are in the nucleotide-binding region of the polymerase.

In certain embodiments, polymerases have mutations that reduce discrimination against ETFD-labelled terminators.

In certain embodiments, DNA polymerases possess exonuclease activity that may allow them to remove incorporated 3′-deoxynucleotide terminators. In certain embodiments, a polymerase bearing one or more mutations or deletions may have reduced 3′-5′ exonuclease activity. In certain embodiments, such mutations or deletions are made in the amino-terminal region of the DNA polymerase. Certain examples of such mutations are described, e.g., in U.S. Pat. No. 4,795,699; U.S. Pat. No. 5,541,099; and U.S. Pat. No. 5,489,523; which are herein incorporated by reference for any purpose. In certain embodiments, such mutations or deletions are made in the region of DNA polymerase that confers 3′-5′ exonuclease activity. In certain embodiments, that region is located from about amino acid 1 to about amino acid 272 of the DNA polymerase.

In certain embodiments, the phenylalanine at position 667 of Thermus aquaticus DNA polymerase is replaced with a tyrosine. A detailed description of this mutant can be found, e.g., in U.S. Pat. No. 5,614,365, which is herein incorporated by reference for any purpose. This mutant polymerase may conveniently be referred to as Thermus aquaticus F667Y, or Taq F667Y (because the phenylalanine (F) at position 667 is replaced with a tyrosine(Y)).

In certain embodiments, the glycine at position 46 of Thermus aquaticus DNA polymerase is replaced with an aspartic acid. This mutant polymerase may conveniently be referred to as Thermus aquaticus G46D, or Taq G46D.

According to certain embodiments, polymerases include, but are not limited to, AmpliTaq FS, Thermus thermophilus HB8, Thermus oshimai, Thermus scotoductus, Thermus thermophilus 1B21, and Thermus thermophilus GK24.

AmpliTaq FS is described, e.g., in U.S. Pat. No. 5,614,365, which is herein incorporated by reference for any purpose. AmpliTaq FS has a mutation at position 46, where a glycine is replaced with an aspartic acid (G46D), and a mutation at position 667, where a phenylalanine is replaced with a tyrosine (F667Y). Certain embodiments comprise a mutant AmpliTaq FS that has the G46D and F667Y mutations of AmpliTaq FS, and additionally has a mutation at position 681, where the glutamine residue is replaced with an isoleucine. This mutant AmpliTaq FS is referred to as AmpliTaq FS (E681I). Certain embodiments comprise a mutant AmpliTaq FS that has the G46D and F667Y mutations of AmpliTaq FS and has two additional mutations as follows: the threonine residue at position 664 is replaced with an asparagine and the arginine residue at position 660 is replaced with a glycine. This mutant AmpliTaq FS is referred to as AmpliTaq FS (T664N; R660G).

Thermus thermophilus HB8 is described, e.g., in U.S. Pat. No. 5,789,224, which is herein incorporated by reference for any purpose. Certain embodiments comprise a mutant Thermus thermophilus HB8 that has three mutations as follows: the aspartic acid residue at position 18 is replaced with an alanine; the phenylalanine residue at position 669 is replaced with a tyrosine; and the glutamine at position 683 is replaced with an arginine. This mutant Thermus thermophilus HB8 is referred to as Thermus thermophilus HB8 (D18A; F669Y; E683R).

Certain embodiments comprise a Thermus thermophilus HB8 that has an amino-terminal deletion that removes the first 271 amino acids of the protein, and that has two mutations as follows: the phenylalanine residue at position 669 is replaced with a tyrosine and the glutamine at position 683 is replaced with a tryptophan. This mutant Thermus thermophilus HB8 is referred to as Thermus thermophilus HB8 (Δ271; F669Y; E683W).

Certain embodiments comprise a mutant Thermus thermophilus HB8 that has two mutations as follows: the aspartic acid residue at position 18 is replaced with an alanine and the phenylalanine residue at position 669 is replaced with a tyrosine. This mutant Thermus thermophilus HB8 is referred to as Thermus thermophilus HB8 (D18A; F669Y).

Thermus oshimai is described, e.g., in U.S. Provisional Application No. 60/334,798, filed Nov. 30, 2001, which is herein incorporated by reference for any purpose. Certain embodiments comprise a mutant Thermus oshimai that has two mutations as follows: the glycine residue at position 43 is replaced with an aspartic acid and the phenylalanine residue at position 665 is replaced with a tyrosine. This mutant of Thermus oshimai is referred to as Thermus oshimai (G43D; F665Y).

Thermus thermophilus 1B21 is described, e.g., in U.S. Provisional Application No. 60/336,046, filed Nov. 30, 2001, which is herein incorporated by reference for any purpose. Certain embodiments comprise a mutant Thermus thermophilus 1B21 that has two mutations as follows: the glycine residue at position 46 is replaced with an aspartic acid and the phenylalanine residue at position 669 is replaced with a tyrosine. This mutant of Thermus thermophilus 1B21 is referred to as Thermus thermophilus 1B21 (G46D; F669Y).

Thermus scotoductus is described, e.g., in U.S. Provisional Application No. 60/334,489, filed Nov. 30, 2001, which is herein incorporated by reference for any purpose. Certain embodiments of the invention comprise a mutant Thermus scotoductus that has two mutations as follows: the glycine residue at position 46 is replaced with an aspartic acid and the phenylalanine residue at position 668 is replaced with a tyrosine. This mutant of Thermus scotoductus is referred to as Thermus scotoductus (G46D; F668Y).

In certain embodiments, a composition comprises at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G).

In certain embodiments, the at least two polymerases are two polymerases. The two polymerases may be present in a composition at any ratio. In certain embodiments, the two polymerases are present in a composition at a ratio of 1:99. In certain embodiments, the ratio is 10:90. In certain embodiments, the ratio is 20:80. In certain embodiments, the ratio is 30:70. In certain embodiments, the ratio is 40:60. In certain embodiments, the ratio is 50:50.

In certain embodiments, a composition comprises Thermus thermophilus GK24 (G46D; F669Y) and Thermus oshimai (G43D; F665Y).

In certain embodiments, a composition comprises at least three polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G). In certain embodiments, the at least three polymerases are three polymerases. The three polymerases may be present in a composition at any ratio.

In certain embodiments, the three polymerases are AmpliTaq FS (E681I), AmpliTaq FS (T664N; R660G), and at least one polymerase selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), and Thermus thermophilus GK24 (G46D; F669Y).

In certain embodiments, the combination of AmpliTaq FS (E681I) and AmpliTaq FS (T664N; R660G) is referred to as FS-I/FS-NG. The ratio of AmpliTaq FS (E681I) and AmpliTaq FS (T664N; R660G) in FS-I/FS-NG may be any ratio. In certain embodiments, the ratio of AmpliTaq FS (E681I) and AmpliTaq FS (T664N; R660G) in FS-I/FS-NG is 2:1. In certain embodiments, the ratio of AmpliTaq FS (E681I) and AmpliTaq FS (T664N; R660G) in FS-I/FS-NG is 99:1. In certain embodiments, the ratio of AmpliTaq FS (E681I) and AmpliTaq FS (T664N; R660G) in FS-I/FS-NG is 90:10. In certain embodiments, the ratio of AmpliTaq FS (E681I) and AmpliTaq FS (T664N; R660G) in FS-I/FS-NG is 80:20. In certain embodiments, the ratio of AmpliTaq FS (E681I) and AmpliTaq FS (T664N; R660G) in FS-I/FS-NG is 70:30. In certain embodiments, the ratio of AmpliTaq FS (E681I) and AmpliTaq FS (T664N; R660G) in FS-I/FS-NG is 60:40. In certain embodiments, the ratio of AmpliTaq FS (E681I) and AmpliTaq FS (T664N; R660G) in FS-I/FS-NG is 50:50. In certain embodiments, the ratio of AmpliTaq FS (E681I) and AmpliTaq FS (T664N; R660G) in FS-I/FS-NG is 40:60. In certain embodiments, the ratio of AmpliTaq FS (E681I) and AmpliTaq FS (T664N; R660G) in FS-I/FS-NG is 30:70. In certain embodiments, the ratio of AmpliTaq FS (E681I) and AmpliTaq FS (T664N; R660G) in FS-I/FS-NG is 20:80. In certain embodiments, the ratio of AmpliTaq FS (E681I) and AmpliTaq FS (T664N; R660G) in FS-I/FS-NG is 10:90. In certain embodiments, the ratio of AmpliTaq FS (E681I) and AmpliTaq FS (T664N; R660G) in FS-I/FS-NG is 1:99.

In certain embodiments, FS-I/FS-NG is combined with at least one polymerase selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), and Thermus thermophilus GK24 (G46D; F669Y). In certain embodiments, FS-I/FS-NG is combined with one polymerase selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), and Thermus thermophilus GK24 (G46D; F669Y).

FS-I/FS-NG and the polymerase selected from Thermus thermophilus HB8 (D18A; F669Y; E683R), Thermus oshimai (G43D; F665Y), Thermus thermophilus HB8 (A271; F669Y; E683W), Thermus thermophilus HB8 (D18A; F669Y), Thermus scotoductus (G46D; F668Y), Thermus thermophilus 1B21 (G46D; F669Y), and Thermus thermophilus GK24 (G46D; F669Y) may be combined at any ratio. In certain embodiments, FS-I/FS-NG and the polymerase selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), and Thermus thermophilus GK24 (G46D; F669Y) are combined at a ratio of 1:99. In certain embodiments, the ratio is 10:90. In certain embodiments, the ratio is 20:80. In certain embodiments, the ratio is 30:70. In certain embodiments, the ratio is 40:60. In certain embodiments, the ratio is 50:50.. In certain embodiments, the ratio is 60:40. In certain embodiments, the ratio is 70:30. In certain embodiments, the ratio is 80:20. In certain embodiments, the ratio is 90:10. In certain embodiments, the ratio is 99:1.

In certain embodiments, a composition comprises FS-I/FS-NG and Thermus oshimai (G43D; F665Y). In certain embodiments, a composition comprises FS-I/FS-NG and Thermus thermophilus GK24 (G46D; F669Y).

In certain embodiments, a composition comprises more than three polymerases selected from Thermus thermophilus HB8 (D18A; F669Y; E683R), Thermus oshimai (G43D; F665Y), Thermus thermophilus HB8 (A271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G).

In certain embodiments, the sequence of a nucleic acid may be determined by generating primer extension products. For example, in certain embodiments, one may employ the method of Sanger (see, e.g., Sanger et al. Proc. Nat. Acad. Sci 74: 5463-5467 (1977)). According to certain embodiments, the present invention provides methods for sequencing a target nucleic acid template using at least two polymerases. In certain embodiments, the methods employ a composition comprising at least one target nucleic acid template, at least one primer, at least one extendable nucleotide, at least one terminator, and at least two polymerases. In certain embodiments, a duplex (double stranded polynucleotide) is formed between a target nucleic acid template and a primer in the composition. In certain embodiments, the primer hybridizes to a predetermined location on the target nucleic acid template.

The composition is incubated under appropriate reaction conditions, such that one or more extendable nucleotides are incorporated sequentially onto the 3′ end of the primer. In certain embodiments, a terminator may be incorporated into the primer extension product, and once incorporated, prevents further incorporation of nucleotides to the 3′ end of the primer extension product by polymerase. In certain embodiments, the primer extension products generated by the primer extension reaction may then be separated based on size. In certain embodiments, the sequence of the nucleic acid template may be determined from the particular sizes of the products and the identity of the terminator on each product.

In certain embodiments, a composition of the invention comprises at least two polymerases, at least one extendable nucleotide, and at least one terminator. In certain embodiments, the at least one extendable nucleotide is selected from dATP, dCTP, dITP, dGTP, dUTP, and dTTP. In certain embodiments, the composition comprises extendable nucleotides dATP, dCTP, dITP, and dUTP. In certain embodiments, the composition comprises extendable nucleotides dATP, dCTP, dITP, and dTTP. In certain embodiments, the at least one terminator is selected from A terminators, C terminators, G terminators, and T terminators. In certain embodiments, the at least one terminator further comprises a label. In certain embodiments, the at least one terminator further comprises an energy-transfer fluorescent dye (ETFD) label. In certain embodiments, the composition .comprises an A terminator, a C terminator, a G terminator, and a T terminator. In certain embodiments, each of the different terminators further comprises a detectably different label. In certain embodiments, each of the different terminators further comprises a detectably different ETFD label. In certain embodiments, the composition contains four different ETFD-labeled terminators, e.g., an ETFD-labeled A terminator, an ETFD-labeled C terminator, an ETFD-labeled G terminator, and an ETFD-labeled T terminator, where each ETFD is detectably different.

In certain embodiments, a composition further comprises at least one buffering agent. In certain embodiments, the at least one buffering agent is selected from Tris and Tricine. In certain embodiments, a composition further comprises at least one type of divalent cation. In certain embodiments, the at least one type of divalent cation is selected from Mg²⁺ and Mn²⁺ . In certain embodiments, a composition further comprises at least one additive. In certain embodiments, the at least one additive is selected from glycerol and DMSO.

In certain embodiments, a composition comprises 80 mM Tris having a pH in the range of 8-9; 5 mM MgCl₂; 0-10% glycerol; 200 μM dATP; 200 μM dCTP; 300 μM dITP; 200 μM dUTP; 25 nM-1225 nM of each of an ETFD-labeled A terminator, an ETFD-labeled C terminator, an ETFD-labeled G terminator, and an ETFD-labeled T terminator; and 1.5-60 units of each of at least two polymerases in a 20 μl reaction volume. In certain embodiments, the composition further comprises thermoplasma acidophilum inorganic pyrophosphatase (TAP). In certain embodiments, one uses the buffer, extendable nucleotides, and terminators from the ABI PRISM BigDye™ Terminators v. 3.0 Cycle Sequencing Kit (Applied Biosystems, Cat. No. 4390236), and replaces the kit's polymerase with at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G).

In certain embodiments, methods are provided for sequencing a target nucleic acid template. In certain embodiments, such methods comprise: forming a composition comprising a target nucleic acid template, at least one primer, at least one extendable nucleotide, at least one terminator, and at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G); and incubating the composition under appropriate conditions to generate at least one primer extension product.

In certain embodiments, the methods include cycle sequencing, in which, following the primer extension reaction and termination, the primer extension product is released from the target nucleic acid template, and a new primer is annealed, extended, and terminated. Cycle sequencing is but one example of amplification of primer extension products. In certain embodiments, cycle sequencing is performed using a thermocycler apparatus. Certain cycle sequencing reactions are described, e.g., in U.S. Pat. No. 5,741,640; U.S. Pat. No. 5,741,676; U.S. Pat. No. 5,756,285; U.S. Pat. No. 5,674,679; and U.S. Pat. No. 5,998,143; which are herein incorporated by reference for any purpose.

In cycle sequencing, an incubation cycle comprises two or more incubations, each incubation comprising a certain temperature for a certain period of time. In certain embodiments, one such incubation cycle comprises 95° C. for 20 seconds, followed by 50° C. for 15 seconds, followed by 60° C. for 4 minutes. In certain embodiments, cycle sequencing comprises repeating the incubation cycle 25 times.

In certain embodiments, the primer extension products may be separated by a mobility-dependent analysis technique, or MDAT. In certain embodiments, the MDAT is electrophoresis. In certain embodiments, by separating the primer extension products, one can determine the sequence of the template nucleic acid based on the size of each product and the identity of the terminator at its 3′ end. In certain embodiments, when the terminator is a labeled terminator, the identity of the terminator at the 3′ end is determined by the identity of the label.

Kits

The invention also provides kits. In certain embodiments, kits serve to expedite the performance of the methods of interest by assembling two or more components used to carry out the methods. In certain embodiments, kits contain components in pre-measured unit amounts to minimize the need for measurements by end-users. In certain embodiments, kits include instructions for performing one or more methods. In certain embodiments, the kit components are optimized to operate in conjunction with one another.

In certain embodiments, kits comprise at least two polymerases selected from Thermus thermophilus HB8 (D18A; F669Y; E683R), Thermus oshimai (G43D; F665Y), Thermus thermophilus HB8 (Δ271; F669Y; E683W), Thermus thermophilus HB8 (D18A; F669Y), Thermus scotoductus (G46D; F668Y), Thermus thermophilus 1B21 (G46D; F669Y), Thermus thermophilus GK24 (G46D; F669Y), AmpliTaq FS (E681I), and AmpliTaq FS (T664N; R660G).

In certain embodiments, the kits may be used to sequence at least one target nucleic acid template. In certain embodiments, the kits further comprise at least one terminator. In certain embodiments, the at least one terminator is a labeled terminator. In certain embodiments, the at least one terminator is selected from an ETFD-labeled A terminator, an ETFD-labeled C terminator, an ETFD-labeled G terminator, and an ETFD-labeled T terminator. In certain embodiments, kits may contain additional components, including, but not limited to, at least one primer and/or at least one extendable nucleotide. In certain embodiments, kits may also include reagents for performing a control reaction, which may include one or more of the above components, and at least one target nucleic acid template.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A composition comprising Thermus thermophilus GK24 (G46D; F669Y) and Thermus oshimai (G43D; F665Y).

2. A method of sequencing a target nucleic acid template comprising:
(a) forming a composition comprising a target nucleic acid template, at least one primer, at least one extendable nucleotide, at least one terminator, Thermus thermophilus GK24 (G46D; F669Y) and Thermus oshimai (G43D; F665Y);
(b) incubating the composition under appropriate conditions to generate at least one primer extension product;
(c) separating the at least one primer extension product, wherein the separating comprises at least one mobility-dependent analysis technique (MDAT);
(d) detecting the at least one primer extension product; and
(e) determining the sequence of the target nucleic acid template.

3. A kit comprising Thermus thermophilus GK24 (G46D; F669Y) and Thermus oshimai (G43D; F665Y).
